(19) United States
(12) Patent Application Publication
(10) Pub. No.: US 2015/0297849 A1
(43) Pub. Date: Oct. 22, 2015

(54) AN APPARATUS AND A METHOD FOR SUPPORTING AN AIRWAY OF A SUBJECT

(71) Applicant: KONINKLIJKE PHILIPS N.V., EINDHOVEN (NL)

(72) Inventors: Meng SHEN, Shanghai (CN); Sheng JIN, Shanghai (CN); Han Yan GONG, Shanghai (CN); Hua JIN, Shanghai (CN); Jianliang WU, Shanghai (CN)

(21) Appl. No.: 14/418,459

(22) PCT Filed: Jul. 31, 2013
(86) PCT No.: PCT/IB2013/056284

§ 371 Date: Jan. 30, 2015

(30) Foreign Application Priority Data
Aug. 3, 2012 (CN) PCT/CN2012/079621

(51) Int. Cl. A61M 16/00; A61B 7/04; A61B 5/00; A61B 7/00; A61M 16/06; A61M 16/20

(52) U.S. Cl. CPC A61M 16/0051; A61M 16/0069; A61M 16/0666; A61M 16/202; A61B 5/4818; A61B 5/4836; A61B 5/7282; A61M 16/0003; A61B 7/003; A61B 7/04; A61M 2205/0272; A61M 2205/3303; A61M 2205/50; A61M 2230/40; A61M 2205/3334

(57) ABSTRACT

The present invention provides an apparatus and a method for supporting an airway of a subject. An aspect of the present invention proposes an apparatus (10) for supporting an airway of a subject, comprising a body (110) configured to enclose one or more external orifices of the airway of the subject, wherein the body forms at least one flow path between the one or more external orifices and the ambient atmosphere; an electrical valve (120) disposed in the flow path for exhalation, wherein the flow resistance of the electrical valve is kept constant during inhalation and can be adjusted during exhalation; a sensor (130) configured to sense snoring of the subject and output a corresponding signal; and a control module (140) connected to the sensor, wherein the control module is configured to sample the signal outputted from the sensor and detect the presence and level of snoring based on the sampled signal at predetermined time points, control the electrical valve to be triggered from a low flow resistance to a high flow resistance which is maintained until the next predetermined time point for supporting the airway of the subject once it has been detected that snoring is present at a certain predetermined time point, and control the electrical valve to adjust the flow resistance in accordance with the presence and level of snoring at the following predetermined time points to obtain a best-fit flow resistance for the subject. Thus, the flow resistance of the electrical valve is automatically fine-tuned to obtain a best-fit flow resistance in accordance with the presence and level of snoring during exhalation, so that the comfort of a subject can be greatly improved.

### FIELD OF THE INVENTION

The invention relates to the field of mitigation of snoring, and particularly to an apparatus and a method for supporting an airway of a subject.

### BACKGROUND OF THE INVENTION

Snoring is related to narrowing or obstruction of the upper airway during sleep (sleep disordered breathing). It is believed to affect as many as 50% of adults including both men and women, i.e. over 45 million Americans. Almost one out of every two American adults snores occasionally, and one in four snores regularly. Snoring poses an inconvenience with regard to the disruption of the sleep-cycle of the sufferer and/or his/her partner, which leads to daytime lethargy and drowsiness and results in a substantial decrease in the quality of life of the sufferer and/or his/her partner.

An approach to mitigate snoring uses airway resistors that resist the flow of exhalation from a subject, thereby supporting the airway during exhalation. However, conventional airway resistors either play a constant role in the case of fixed flow resistance or just adjust flow resistance in a very simple way during exhalation. For example, international patent application WO 2010/076704A1 discloses a respiration appliance configured to support an airway of a subject, which, on the one hand, keeps the exhalation flow resistance at a low value when it is determined that snoring does not occur, and which, on the other hand, increases the exhalation flow resistance to support the airway of the subject when it is determined that snoring occurs. In some cases, conventional airway resistors that are considered to be uncomfortable by a subject may not provide adequate support for a subject.

### SUMMARY OF THE INVENTION

It is an object of the present invention to provide an improved apparatus and method for supporting an airway of a subject.

According to one aspect of the present invention, an apparatus is provided for supporting an airway of a subject, comprising:

a body configured to enclose one or more external orifices of the airway of the subject, wherein the body forms at least one flow path between the one or more external orifices and the ambient atmosphere;

an electrical valve disposed in the flow path for exhalation, wherein the flow resistance of the electrical valve is kept constant during inhalation and can be adjusted during exhalation;

a sensor configured to sense snoring of the subject and output a corresponding signal; and

a control module connected to the sensor, wherein the control module is configured to sample the signal outputted from the sensor and detect the presence and level of snoring based on the sampled signal at predetermined time points, control the electrical valve to be triggered from a low flow resistance to a high flow resistance which is maintained until the next predetermined time point for supporting the airway of the subject once it has been detected that snoring is present at a certain predetermined time point, and control the electrical valve to adjust the flow resistance in accordance with the presence and level of snoring at the following predetermined time points to obtain a best-fit flow resistance for the subject.

Since the apparatus for supporting an airway of a subject is capable of automatically fine-tuning the flow resistance of the electrical valve to obtain a best-fit flow resistance in accordance with the presence and level of snoring during exhalation, the comfort for a subject can be greatly improved.

In accordance with an embodiment of the present invention, the adjusting of the flow resistance in accordance with the presence and level of snoring at the following predetermined time points comprises:

decreasing the flow resistance to a lower value at the following predetermined time points, when it is detected that snoring is not present;

keeping the flow resistance stable at the following predetermined time points, when it is detected that the level of snoring is lower than the previous level of snoring; and

increasing the flow resistance to a higher value at the following predetermined time points, when it is detected that the level of snoring is higher than the previous level of snoring.

In accordance with a further embodiment of the present invention, if the flow resistance reaches the maximum and snoring is still present, the apparatus for supporting an airway of a subject gives an indication to show that it is not suitable for the subject in question.

In accordance with an embodiment of the present invention, to save power for prolonging the battery life of the apparatus, preferably the time interval between two successive predetermined time points can be varied in dependence on the operation of the control module.

In accordance with a further embodiment of the present invention, the time interval is set to a predetermined value before the control module detects that snoring is present, and the time interval is reduced to less than the predetermined value when the control module detects that snoring is present, and the time interval is restored to the predetermined value when the control module detects that snoring is not present anymore.

In a preferred embodiment of the present invention, the control module does not perform the sampling and detecting operations in a certain period and the electrical valve maintains the low flow resistance in said certain period after power-on, and after said certain period the control module begins to perform the sampling and detecting operations and begins to control the operation of the electrical valve. Thus, this can help the subject to adjust to changes of the flow resistance.

According to another aspect of the present invention, a method is provided for supporting an airway of a subject, comprising:

providing an electrical valve in a flow path for exhalation between one or more external orifices of the airway of the subject and the ambient atmosphere, wherein the flow resistance of the electrical valve is kept constant during inhalation and can be adjusted during exhalation;

detecting the presence and level of snoring of the subject at predetermined time points;

controlling the electrical valve to be triggered from a low flow resistance to a high flow resistance which is maintained until the next predetermined time point for supporting the airway of the subject, once it has been detected that snoring is present at a certain predetermined time point; and

controlling the electrical valve to adjust the flow resistance in accordance with the presence and level of snoring at the following predetermined time points to obtain a best-fit flow resistance for the subject.

Other objects and advantages of the present invention will become more apparent and will be easily understood with reference to the description made in combination with the accompanying drawings.

### DESCRIPTION OF THE DRAWINGS

The present invention will be described and explained hereinafter in more detail by means of embodiments and with reference to the drawings, in which:

FIG. 1 illustrates an apparatus for supporting an airway of a subject in accordance with an embodiment of the present invention;

FIG. 2 illustrates an electrical valve in accordance with an embodiment of the present invention;

FIG. 3 illustrates a flowchart of the method of supporting an airway of a subject in accordance with an embodiment of the present invention.

The same reference signs in the figures indicate similar or corresponding features and/or functionalities.

### DETAILED DESCRIPTION

The embodiments of the present invention will be described hereinafter in more detail with reference to the drawings.

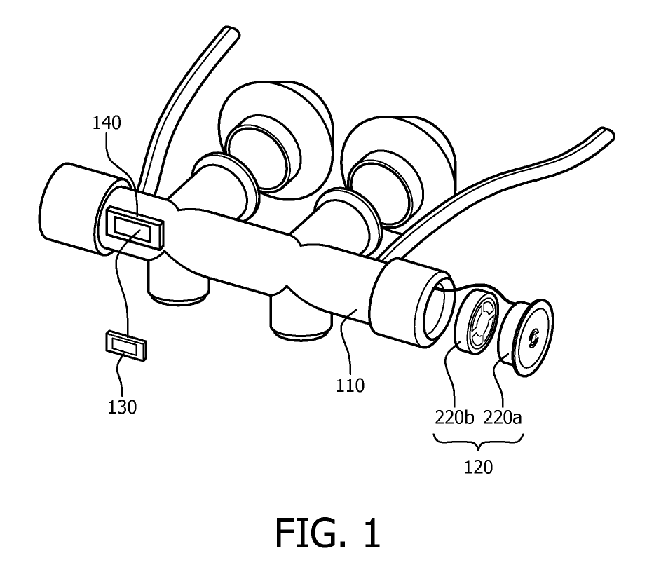

Referring to FIG. 1, said figure shows an apparatus 10 for supporting an airway of a subject in accordance with an embodiment of the present invention. In particular, the apparatus 10 includes a body 110 that encloses one or more external orifices of the airway of the subject, an electrical valve 120, a sensor 130 and a control module 140. The body 110 forms at least one flow path between the one or more external orifices and the ambient atmosphere, and the electrical valve 120 is disposed in a flow path for exhalation. On the one hand, the flow resistance of the electrical valve 120 can be adjusted to regulate the flow of gas from the one or more external orifices of the airway of the subject to the ambient atmosphere during exhalation, and, on the other hand, the flow resistance of the electrical valve 120 is kept constant during inhalation.

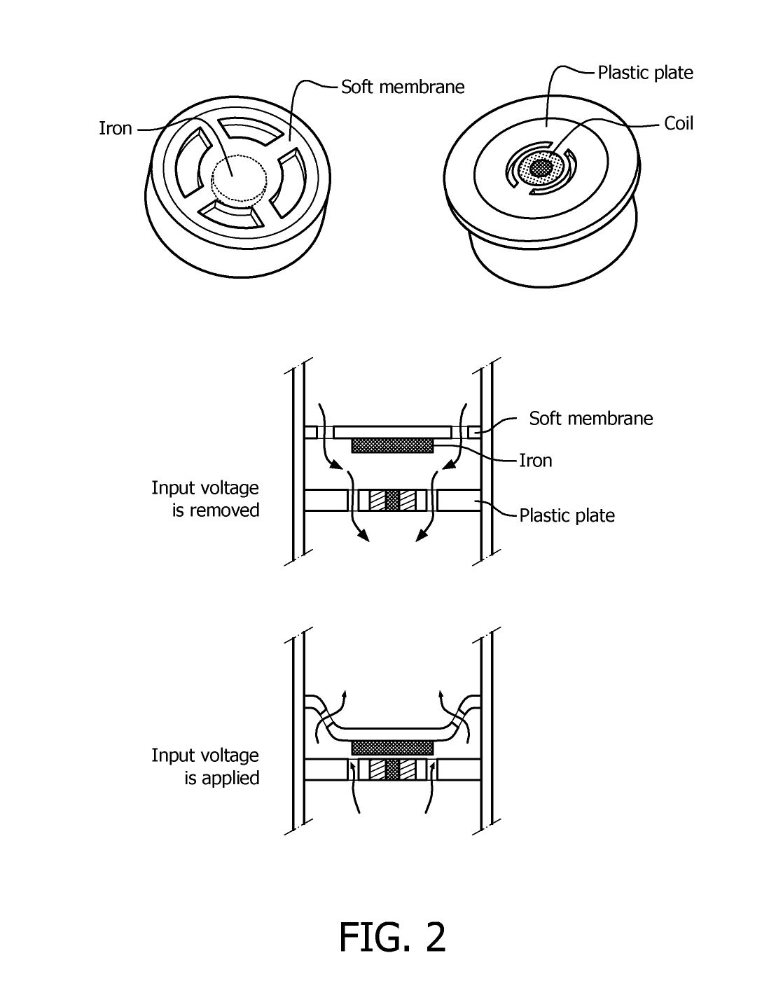

Referring now to FIG. 2, said figure shows an electrical valve 120 in accordance with an embodiment of the present invention. In this embodiment, the electrical valve 120 is an electromagnetic valve, which may comprise a first component 220a and a second component 220b. The first component 220a acts as a source for magnetic field generation as a function of the input voltage. Accordingly, it comprises a thick plastic plate with a plurality of hollow structures and a coil made of copper wires with a magnetic core formed in the central one of the plurality of hollow structures, wherein the magnetic core helps to concentrate the magnetic flux and hence enhances the magnetic field. The second component 220b has a soft membrane with embedded soft iron in which a plurality of openings exist for allowing gas to pass. Further, the electromagnetic valve also may comprise a plastic housing for assembling the first component 220a and the second component 220b in the body 110 of the apparatus 10.

As shown in FIG. 2, when the input voltage is applied to the first component 220a, an attractive force is generated between the soft membrane structure in the second component 220b and the coil structure in the first component 220a, and thus the soft membrane structure deflects towards the surface of the plastic plate of the first component 220a. The soft membrane structure can be kept at different stable deflecting positions, so that the valve has a different flow resistance if the generated attractive force can be counteracted by the flexible force of the soft membrane structure than when the soft membrane structure touches the surface of the plastic plate of the first component 220a, so that the valve is completely closed if the generated attractive force cannot be counteracted by the flexible force of the soft membrane structure. The deflected soft membrane structure will automatically resume its original position when the input voltage is removed.

However, it should be understood that the present invention is not limited to this embodiment and can also employ a piezoelectric valve or any other suitable electrical valve.

Referring to FIG. 1 again, the control module 140 is connected to the sensor 130 that senses parameters indicative of the state of snoring of the subject and outputs a corresponding signal. In one illustrative example, the sensor 130 may be an adhesive piezoelectric sensor provided on the subject, which senses vibration of tissues around the airway of the subject caused by snoring and converts the vibration into an electrical output signal. In another illustrative example, it is preferable that the sensor 130 may be a microphone which is capable of sensing snoring sound and converting the snoring sound into an electrical output signal. The subject may feel more comfortable because the microphone does not have to be provided on the subject.

Further, the control module 140 is configured to sample the signal outputted from the sensor 130 and detect the presence and level of snoring based on the sampled signal at predetermined time points. For example, if a microphone is employed as the sensor and it does not sense snoring sound, the output signal thereof is zero and accordingly the sampled signal is zero as well. This means that snoring is not present. On the other hand, if the microphone senses snoring sound, the output signal thereof is larger than zero and accordingly the sampled signal is larger than zero as well. This means that snoring is present, and the magnitude of the sampled signal represents the level of snoring.

When the control module 140 detects that snoring is present at a certain predetermined time point, the electrical valve 120 is triggered from a low flow resistance to a high flow resistance under the control of the control module 140, and the high flow resistance is maintained until the next predetermined time point in order to support the airway of the subject. Thereafter, the flow resistance of the electrical valve 120 is adjusted in accordance with the presence and level of snoring under the control of the control module 140 at the following predetermined time points, so that a best-fit flow resistance for the subject can be obtained.

In one embodiment, the control module 140 may adjust the flow resistance of the electrical valve 120 at the following predetermined time points in the following manner. In particular, the control module 140 decreases the flow resistance of the electrical valve 120 to a lower value when it detects that snoring is not present at the following predetermined time points. Furthermore, the control module 140 keeps the flow resistance of the electrical valve 120 stable at the following predetermined time points when it detects that the level of snoring is lower than the previous level of snoring, while the control module 140 increases the flow resistance of the electrical valve 120 to a higher value at the following predetermined time points when it detects that the level of snoring is higher than the previous level of snoring.

Sometimes, even when the flow resistance of the electrical valve 120 reaches the maximum, snoring is still present. In this case, the apparatus 10 may indicate that it is not suitable for the subject by any appropriate means such as making a buzzing sound or emitting light.

According to the present invention, a time interval between two adjacent predetermined time points (i.e., the sampling and detecting frequency of snoring) may be varied in dependence on the operation of the control module 140 so as to save power for prolonging the battery life of the apparatus 10. By way of an illustrative example, the time interval may be set to a predetermined value (e.g. 10 min) before the control module 140 detects that snoring is present, and the time interval is reduced to a lower value (e.g. 5 mins) after it has been detected that snoring is present. Then, the time interval may be restored to the predetermined value if the control module 140 detects that snoring is not present anymore.

Furthermore, in order to help the subject adjust to the change of the flow resistance, it is preferable that the control module 140 does not perform the sampling and detecting operations for a certain period of time (e.g. 20 mins) and the electrical valve 120 maintains the low flow resistance in said certain period after power-on. After said certain period, the control module 140 may resume the sampling and detecting operations and control the operation of the electrical valve 120.

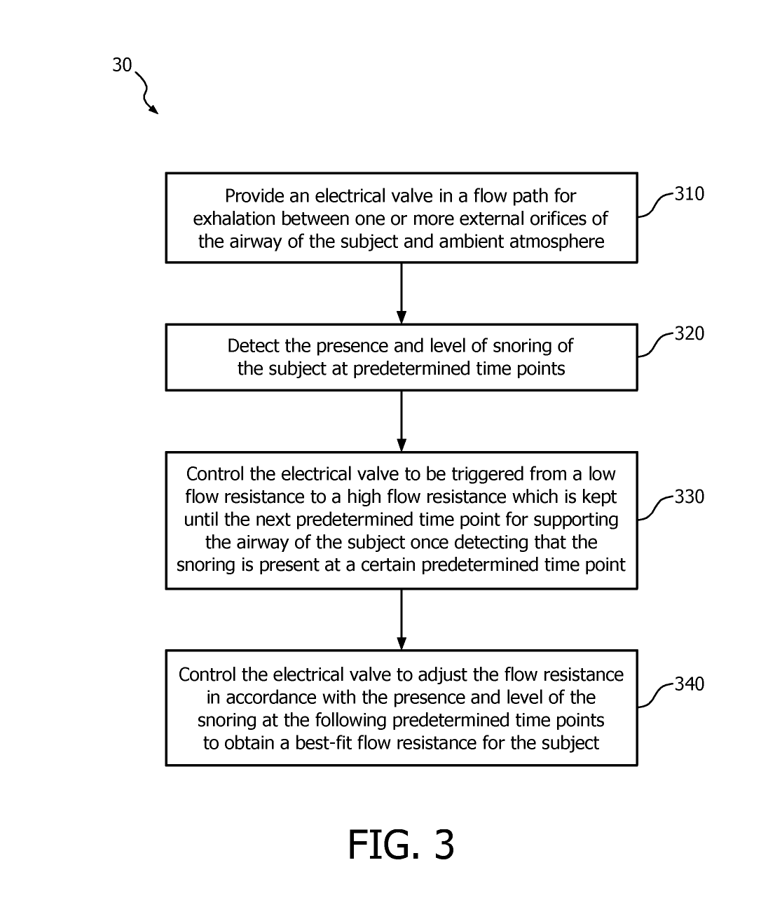

Referring now to FIG. 3, this figure shows a flowchart of the method 30 of supporting an airway of a subject in accordance with an embodiment of the present invention.

As can be seen from FIG. 3, in step 310 an electrical valve 120 is provided in a flow path for exhalation between one or more external orifices of the airway of the subject and the ambient atmosphere. As described above, the flow resistance of the electrical valve 120 is kept constant during inhalation and can be adjusted during exhalation. For example, the electrical valve 120 may be an electromagnetic valve as shown in FIG. 2.

Then, the presence and level of snoring of the subject is detected at predetermined time points in step 320. Next, the electrical valve 120 is controlled to be triggered from a low flow resistance to a high flow resistance which is maintained until the next predetermined time point for supporting the airway of the subject once it has been detected that snoring is present at a certain predetermined time point in step 330. Finally, the electrical valve 120 is controlled to adjust the flow resistance in accordance with the presence and level of snoring at the following predetermined time points to obtain a best-fit flow resistance for the subject in step 340.

In one embodiment, the adjustment of the flow resistance in step 340 may be performed in the following manner. In particular, when it is detected that snoring is not present, the flow resistance is decreased to a lower value at the following predetermined time points. Furthermore, when it is detected that the level of snoring is lower than the previous level of snoring, the flow resistance is kept stable at the following predetermined time points, while, when it is detected that the level of snoring is higher than the previous level of snoring, the flow resistance is increased to a higher value at the following predetermined time points.

Moreover, according to the present invention, if the flow resistance reaches the maximum and snoring is still present, an indication may be given to show that the electrical valve 120 is not suitable for the subject.

In accordance with an embodiment of the present invention, a time interval between two adjacent predetermined time points may be varied in dependence on the detecting operation. For example, the time interval may be set to a predetermined value before it is detected that snoring is present, and the time interval is reduced to less than the predetermined value after it is detected that snoring is present. Then, the time interval may be restored to the predetermined value if it is detected that snoring is not present anymore.

In a preferred embodiment of the present invention, the electrical valve 120 maintains the low flow resistance in a certain period after power-on, and the operations of sampling, detecting and controlling are not performed in said certain period. After this period, these operations are initiated. This can help the subject adjust to change of the flow resistance.

It should be noted that the above-mentioned embodiments illustrate rather than limit the invention and that those skilled in the art will be able to design alternative embodiments without departing from the scope of the appended claims. In the claims, any reference signs placed between parentheses shall not be construed as limiting the claim. The word “comprising” does not exclude the presence of elements or steps not listed in a claim or in the description. The word “a” or “an” preceding an element does not exclude the presence of a plurality of such elements. The usage of the words first and second, et cetera, does not indicate any ordering. These words are to be interpreted as names.

1. An apparatus for supporting an airway of a subject comprising:
a body configured to enclose one or more external orifices of the airway of the subject, wherein the body forms at least one flow path between the one or more external orifices and the ambient atmosphere;
an electrical valve disposed in the flow path for exhalation, wherein the flow resistance of the electrical valve is kept constant during inhalation and can be adjusted during exhalation;
a sensor configured to sense snoring of the subject and output a corresponding signal;
a control module connected to the sensor, wherein the control module is configured to sample the signal outputted from the sensor and detect the presence and level of snoring based on the sampled signal at predetermined time points, control the electrical valve to be triggered from a low flow resistance to a high flow resistance which is maintained until the next predetermined time point for supporting the airway of the subject once it has been detected that 1 snoring is present at a certain predetermined time point, and control the electrical valve to adjust the flow resistance in accordance with the presence and level of snoring at the following predetermined time points to obtain a best-fit flow resistance for the subject;
wherein the electrical valve is an electromagnetic valve which comprises a first component having a thick plastic plate with a plurality of hollow structures and a coil made of copper wires with a magnetic core formed in the central one of the plurality of hollow structures; a second component having a soft membrane with embedded soft iron in which a plurality of openings are present for allowing gas to pass; and a plastic housing for assembling the first component and the second component in the body.

2. The apparatus of claim 1, wherein the sensor is a microphone for sensing snoring sound.

3. (canceled)

4. The apparatus of claim 1, wherein the adjusting of the flow resistance in accordance with the presence and level of snoring at the following predetermined time points comprises:
decreasing the flow resistance to a lower value at the following predetermined time points when it is detected that snoring is not present;
keeping the flow resistance stable at the following predetermined time points when it is detected that the level of snoring is lower than the previous level of snoring; and
increasing the flow resistance to a higher value at the following predetermined time points when it is detected that the level of snoring is higher than the previous level of snoring.

5. The apparatus of claim 4, wherein the apparatus gives an indication to show that it is not suitable for the subject if the flow resistance reaches the maximum and snoring is still present.

6. The apparatus of claim 1, wherein a time interval between two adjacent predetermined time points can be varied in dependence on the operation of the control module (140).

7. The apparatus of claim 6, wherein the time interval is set to a predetermined value before the control module detects that snoring is present, the time interval is reduced to less than the predetermined value after the control module detects that snoring is present, and the time interval is restored to the predetermined value after the control module detects that snoring is not present anymore.

8. The apparatus of claim 1, wherein the control module does not perform the sampling and detecting operations in a certain period and the electrical valve maintains the low flow resistance in said certain period after power-on, and after said certain period the control module begins to perform the sampling and detecting operations and begins to control the operation of the electrical valve.

9-15. (canceled)
